(19) United States
(12) Patent Application Publication
(10) Pub. No.: US 2010/0016876 A1
(43) Pub. Date: Jan. 21, 2010

(54) Microsurgical Cutting Arrangement, Especially for Refractive Eye Surgery

(75) Inventor: Christof Donitzky, Eckental (DE)

Correspondence Address: HAYNES AND BOONE, LLP;IP Section, 2323 Victory Avenue, Suite 700, Dallas, TX, 75219, US

(73) Assignee: Wavelight AG, Erlangen (DE)

(21) Appl. No.: 12/063,557

(22) PCT Filed: Aug. 3, 2006
(86) PCT No.: PCT/EP2006/007698

§ 371 Date: Jun. 15, 2009

(30) Foreign Application Priority Data
Aug. 12, 2005 (EP) 05017647.8

(51) Int. Cl. A61F 9/013

(52) U.S. Cl. 606/166

(57) ABSTRACT

A microsurgical cutting arrangement, in particular for refractive eye surgery, includes a cutting blade holder as well as a cutting blade unit (14) that can be inserted into a receptacle of the cutting blade holder. A cutting blade (12) of the cutting blade unit forms a cutting edge (20) on a first (front) blade edge, and is supported via an opposite (rear) second blade edge (24) on linear bearing means (42) of the cutting blade holder. According to the invention the rear blade edge forms at least two bearing points (26,28) spaced from one another for the bearing of the cutting blades on the bearing means, and between each pair of adjacent bearing points is set back with respect to am imaginary straight line joining the relevant bearing points, in the direction of the front blade edge (at 30).

### CROSS REFERENCE

This application was originally filed as Patent Cooperation Treaty Application Number PCT/EP2006/007698 filed Aug. 3, 2006, which claims priority of European Application Number 05017647.8, filed Aug. 12, 2005.

### CROSS-REFERENCE TO RELATED APPLICATION

This application is a United States national phase application of co-pending international patent application number PCT/EP2006/07698, filed Aug. 3, 2006, the disclosure of which is incorporated herein by reference.

### BACKGROUND

The present invention relates to a microsurgical cutting arrangement, in particular for refractive surgery in ophthalmology, with a cutting blade holder and a cutting blade unit that can be inserted into a receptacle of the cutting blade holder, wherein the cutting blade unit comprises a cutting blade with a cutting edge formed on a first blade edge, and wherein the cutting blade holder comprises linear bearing means, on which the cutting blade unit in the correctly inserted state in the receptacle is supported via a second blade edge of the cutting blade opposite the said first blade edge.

### SUMMARY

In the refractive correction of defective vision in the human eye it is known to lift off or detach a surface flap from the cornea by means of a microsurgical cutting instrument termed a microkeratome, so that the flap is still joined on one side to the cornea by a so-called hinge. By lifting up and folding the flap to one side the underlying corneal regions (stroma) are accessible for reshaping of the cornea by means of a laser. After completion of the laser treatment the flap is folded back in position.

The microkeratome normally comprises a suction ring unit that can be placed on the eyeball (limbus), on which a cutting blade holder replacably loaded with a cutting blade can be movably guided. For the flap preparation the cutting blade holder is moved by means of an electric motor drive in a feed direction over the cornea. At the same time the cutting blade with its cutting edge projecting from the cutting blade holder cuts into the cornea and detaches the flap. In addition to the feed movement of the cutting blade holder, the cutting blade normally executes lateral oscillations.

In a known configuration the cutting blade together with its rear blade edge lying opposite the cutting edge is supported on the cutting blade holder. The rear blade edge is in this connection designed as a straight bearing edge. To ensure a precise guidance of the cutting blade in its oscillating sideways movement, the rear bearing edge must be of sufficient length. This results in a correspondingly increased friction of the cutting blade on the cutting blade holder. Given the oscillation frequency of the cutting blade, which is often between 15 and 500 Hz, in particular between 100 and 250 Hz, a high degree of accessibility of the cutting blade in the cutting blade holder is of course very desirable. At such oscillation frequencies the weight of the cutting blade is also an important factor. The weight should be as low as possible so that overall only small masses have to be moved.

The object of the invention is to provide a microsurgical cutting arrangement of the type described in the introduction, which permits an easily accessible and at the same time precise guidance of the cutting blade in the cutting blade holder.

In order to meet this object it is proposed according to the invention that the second blade edge of the cutting blade forms at least two bearing points spaced apart from one another for the bearing of the cutting blade on the bearing means of the cutting blade holder, and between each pair of adjacent bearing points is set back with respect to an imaginary straight line joining the relevant bearing points, in the direction of the first blade edge. Due to the provision of a plurality of bearing points spaced apart from one another on the second (rear) blade edge, the size of the contact region in which the rear blade edge comes into engagement with the bearing means of the cutting blade holder can overall be kept small, while maintaining a precise guidance of the cutting blade. This reduces the friction between the cutting blade and cutting blade holder. The region between the bearing points can advantageously be utilised to reduce the material and thus the weight of the cutting blade if in this region the rear blade edge is set back with respect to the blade interior.

In such generic cutting arrangements the degree to which the cutting edge of the cutting blade projects beyond the cutting blade holder determines the thickness of the corneal flap that is detached. The blade projection depends for its part on the length of the cutting blade from the cutting edge up to the point where the blade is supported at the rear, in other words up to the rear blade edge. Since it is extremely important for the success of an operation that a predetermined, desired flap thickness be precisely maintained, usually very stringent requirements are placed on the accuracy of the blade length. With a single bearing edge that extends over a large part of the blade width, unevennesses in the rectilinearity of the bearing edge as well as a certain lack of parallelism of the bearing edge with respect to the cutting edge cannot be completely excluded. This can lead to differences between the actual flap thickness and the desired flap thickness. The provision of separate, locally bounded bearing points instead of a single continuous bearing edge is therefore advantageous for the maintenance of the required narrow tolerances of the effective blade length.

In a preferred embodiment at least one bearing point of the cutting blade is formed by a rounded section of the second blade edge. Alternatively or in addition at least one bearing point of the cutting blade can be formed by a conically shaped section of the second blade edge. With a rounded or conically shaped configuration of the relevant blade edge section, a bearing point can be created that has a virtually punctiform contact with the bearing means of the cutting blade holder. In this way an extremely high guidance accuracy of the cutting blade for the sideways oscillation can be maintained. It is however also conceivable to form at least one bearing point of the cutting blade from a rectilinear section of the second blade edge. Such a rectilinear bearing point can be very much shorter compared to a single straight bearing edge extending over a large part of the blade width and is therefore considerably less susceptible to any unevennesses in the rectilineararity and lack of parallelism with respect to the cutting edge.

All bearing points of the rear blade edge can have the same basic geometry, and thus for example can be rounded or conically shaped. However, at least two bearing points can also have different geometries. This is conceivable in particular if the second blade edge of the cutting blade forms at least three bearing points. In this case two outer bearing points can have the same basic geometry and a middle bearing point can have a different geometry. It is in principle also conceivable to design all bearing points of the rear blade edge so as to have different geometries.

For an exact positioning of the cutting blade unit it is recommended that, when the cutting blade unit is correctly inserted into the cutting blade holder, resilient tensioning means are active between the said unit and the cutting blade holder, which press the cutting blade at its bearing points against the bearing means of the cutting blade holder.

For a relatively frictionless guidance of the cutting blade on the bearing means of the cutting blade holder it is advantageous if, viewed in a cross-section transverse to the linear extension of the bearing means, the said bearing means have an arcuate, in particular circular arcuate, curved contour.

The invention aims to protect not only a combination of cutting blade holder and cutting blade unit, but also a cutting blade unit per se, as well as the use of a microsurgical cutting arrangement of the type described hereinbefore with a cutting blade holder. The cutting blade unit includes in this connection a cutting blade with a cutting edge formed on a first blade edge, wherein a second blade edge of the cutting blade opposite to the first blade edge forms at least two bearing points arranged spaced from one another for the bearing of the cutting blade on linear bearing points of the cutting blade holder, and between each pair of adjacent bearing points is set back with respect to an imaginary straight line joining the relevant bearing points, in the direction of the first blade edge.

### BRIEF DESCRIPTION OF THE DRAWINGS

The invention is described in more detail hereinafter with the aid of the accompanying drawings, in which:

FIG. 1 is a section through a blade receptacle region of a cutting blade holder according to an example of implementation,

FIG. 2 is a perspective view of an example of implementation of a cutting blade unit suitable for use with the cutting blade holder of FIG. 1, and

FIGS. 3 to 6 show different variants of a cutting blade for the cutting blade unit of FIG. 2.

### DETAILED DESCRIPTION

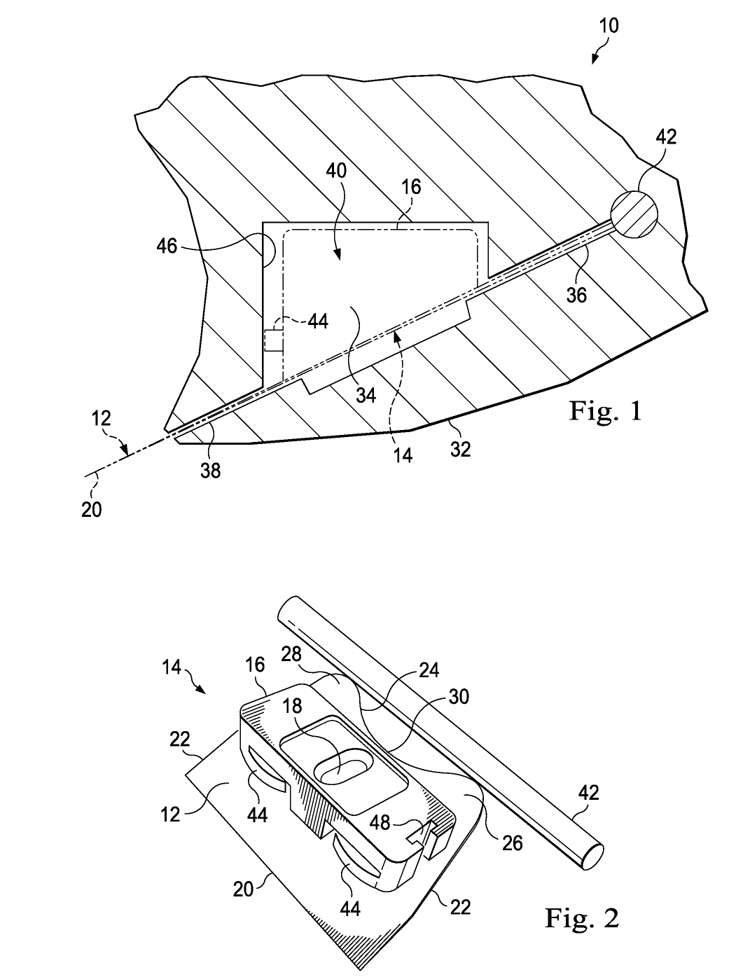

The cutting blade holder shown in section in FIG. 1 and generally identified by the reference numeral 10 is movably guidedly held or can be held in a manner known per se on a suction ring unit of an ophthalmological microkeratome, which is not shown in more detail. After the suction ring unit has been placed on the eye to be operated on and held there under suction by means of a vacuum, the cutting blade holder can be moved by means of an electric motor drive, likewise not shown in more detail, in a feed direction over the cornea of the eye, whereby a cutting blade 12 (FIG. 2) separates a flap from the cornea.

As can be seen in FIG. 2, the cutting blade 12 is part of a cutting blade unit 14, which includes in addition to the actual cutting blade 12 an attachment 16 on one of the flat upper sides of the cutting blade 12. The attachment 16 is firmly connected to the cutting blade 12, preferably by an interlocking-type or frictional-type connection. A material-to-material connection using an adhesive is in principle also possible. The attachment 16 simplifies the manipulation of the cutting blade unit 14. On its free upper side the attachment has an elongated depression 18, in which an eccentric pin of a drive shaft of the aforementioned electric motor drive engages during operation of the microkeratome. The cutting blade unit 14 is thereby caused to execute laterally oscillating movements (transverse to the feed direction).

The cutting blade 12 has a straight front blade edge, which forms a cutting edge 20. Blunt lateral blade edges 22 adjoin the front blade edge and transform in the rear region of the cutting blade 12 into a rear blade edge 24. The rear blade edge 24 is designed having two rounded bearing sections 26, 28 spaced apart from one another, between which is arranged a blade edge section 30 that is set back. The curvature of the rounded blade edge sections 26, 28 can for example at least in part be circular. The blade edge section 30 that is set back is in the illustrated example also rounded, but can also have any other desired shape. The contoured indentation of the cutting blade 12 formed by the set-back blade edge section 30 preferably does not extend further than the attachment 16.

The cutting blade holder 10 comprises a blade holder housing 32, in which is formed a receptacle 34 for the cutting blade unit 14. The receptacle 34 is conveniently open on one side of the cutting blade holder 10, so that the cutting blade unit 14 can be inserted laterally into the receptacle 34 of the cutting blade holder 10 and can be removed therefrom after use. The receptacle 34 has two slit-shaped sections 36, 38, between which is located an enlarged section 40. When the cutting blade unit 14 is inserted into the receptacle 34, the attachment 16 extends into the enlarged section 40, while the blade regions in front and behind the attachment 16 extend into the slit-shaped sections 36, 38 of the receptacle 34. If the cutting blade unit 14 is inserted correctly into the receptacle 34, as indicated by the dotted lines in FIG. 1, then the cutting blade 12 with its cutting edge 20 projects from the cutting blade holder 10. At the same time the cutting blade 12 with its rear bearing sections 26, 28 is supported on a guide bearing rod 42 incorporated in the blade holder housing 32.

On account of the rounded shape of the bearing sections 26, 28 the contact between the cutting blade 12 and the guide bearing rod 42 is virtually punctiform, i.e. there are a total of two bearing points between the cutting blade 12 and the guide bearing rod 42. The punctiform support of the cutting blade 12 on the guide bearing rod 42 ensures a particularly high frictionless contact when the cutting blade holder 14 executes lateral oscillations on operation of the microkeratome and the bearing sections 26, 28 accordingly move along the rectilinear guide bearing rod 42. The punctiform contact is promoted still further by an arcuately curved outer circumferential surface of the guide bearing rod 42. The guide bearing rod 42 is therefore expediently formed from a rod of circular cross-section, as can be seen in particular in FIG. 1. It is understood that alternatively rods of other cross-sectional shapes can be used, for example a rod of oval or elliptical cross-section. Of course, rods that provide a flat bearing surface for the cutting blade 12 can also be employed, for example a rod of rectangular or triangular cross-section.

In FIG. 2 it can be seen that the attachment 16 is designed with two spring tongues 44, which are intended and designed for co-operation with a front boundary wall 46 of the expanded section 40 of the receptacle 34. The spring tongues 44 pretension the cutting blade unit 14 in the rearwards direction, i.e. against the guide bearing rod 42, when the cutting blade unit 14 is correctly inserted into the receptacle 34. The spring tongues 44 can be produced in one part with the attachment 16, but alternatively can be formed from separate elements, which are bonded or welded to the attachment 16 or fastened thereto in some other way. It is understood that arbitrarily shaped spring elements can be used to generate a spring pretensioning that pretensions the cutting blade unit 14 in the direction of the guide bearing rod 26. It is in addition also not necessary for the spring elements (in this case the spring tongues 44) to be arranged on the cutting blade unit 44, as is the case in the example illustrated in FIGS. 1 and 2. The spring elements can just as well be arranged on the cutting blade holder 14 in the receptacle 34 or can project thereinto.

An undercut T-shaped groove 48, with which an actuating rod (not shown in more detail) can be brought into feed-transmitting and tensile force-transmitting engagement, is formed on the side of the attachment 16, which for example can be injection moulded from plastics material but can also be made from metal or a ceramic material. By means of such an actuating rod the cutting blade unit 14 can be inserted without any problem into the receptacle 14 and/or removed from the latter.

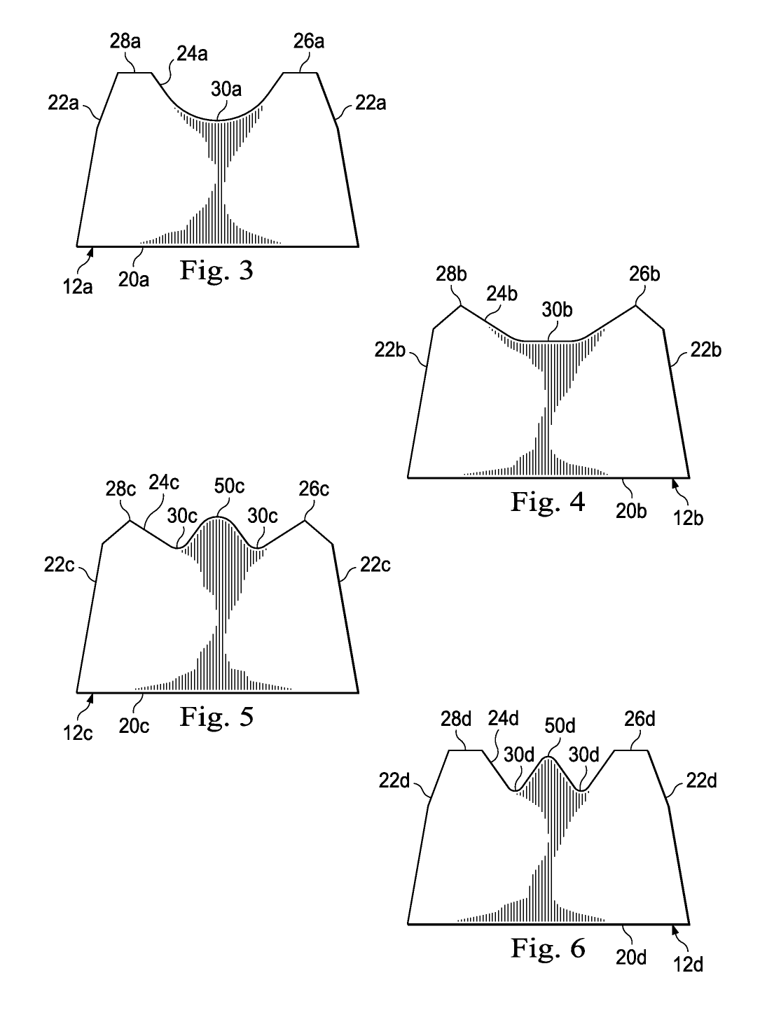

Reference will now be made to the alternative blade contours of the cutting blade illustrated in FIGS. 3 to 6. In these figures components having the same effect are identified by the same reference numerals as in FIG. 2, but with the addition of a lower case letter.

In the variant shown in FIG. 3 the bearing sections 26a, 28a of the cutting blade 12a are formed from short, straight segments of the rear blade edge 24a.

In the variant of FIG. 4 on the other hand the bearing sections 26b, 28b of the cutting blade 12b are of conical shape, so that, like the rounded bearing sections 26, 28 of the cutting blade 12 shown in FIG. 2, a substantially punctiform contact with a rear abutment surface provided in the blade receptacle of the cutting blade holder can be produced.

In the variant shown in FIG. 5, in addition to the two bearing sections 26c, 28c, which in this case too are of conical shape, a third, middle bearing section 50c is provided, which however has a different geometry to the two outer bearing sections 26c, 28c. Specifically, in the example illustrated in FIG. 5 the middle bearing section 50c is rounded, so that, like the two outer bearing sections 26c, 28c, it forms an approximately punctiform contact with the rear abutment surface of the cutting blade holder. A section 30c that is set back is provided on both sides of the middle bearing section 50c.

The variant illustrated in FIG. 6 differs from that of FIG. 5 in that the two outer bearing sections 26d, 28d are formed as short, straight blade edge segments, similar to the case in FIG. 3.

Regardless of the specific geometry of the bearing sections, in the two variants illustrated in FIGS. 5 and 6 the contact points and contact regions of all bearing sections lie on an imaginary straight line, with the result that a precise lateral guidance of the cutting blade is ensured.

What is claimed is:

1. Microsurgical cutting arrangement, in particular for refractive eye surgery, with a cutting blade holder and a cutting blade unit that can be inserted into a receptacle of the cutting blade holder for lateral movement, wherein the cutting blade unit comprises:
a cutting blade with a cutting edge formed on a first blade edge, and a second blade edge opposite the front blade edge; and
wherein the cutting blade holder comprises a linear bearing surface, on which the cutting blade unit when correctly inserted into the receptacle is laterally movably supported via the second blade edge of the cutting blade, characterised in that the second blade edge of the cutting blade forms at least two bearing points spaced apart from one another for the bearing of the cutting blade on the bearing surface of the cutting blade holder and between each pair of adjacent bearing points is set back with respect to an imaginary straight line joining the relevant bearing points, in the direction of the first blade edge.

2. Microsurgical cutting arrangement according to claim 1, characterised in that at least one bearing point of the cutting blade is formed from a rounded section of the second blade edge.

3. Microsurgical cutting unit according to claim 1, characterised in that at least one bearing point of the cutting blade is formed from a conically shaped section of the second blade edge.

4. Microsurgical cutting unit according to claim 1, characterised in that at least one bearing point of the cutting blade is formed from a rectilinear section of the second blade edge.

5. Microsurgical cutting unit according to claim 1, characterised in that at least two bearing points have different geometries.

6. Microsurgical cutting unit according to claim 1, characterised in that the second blade edge of the cutting blade forms at least three bearing points.

7. Microsurgical cutting unit according to claim 1, characterised in that when the cutting blade unit is correctly inserted into the cutting blade holder, resilient pretensioning members are positioned between said unit and the cutting blade holder, to press the cutting blade at its bearing points against the bearing surface of the cutting blade holder.

8. Microsurgical cutting unit according to claim 1, characterised in that the bearing surface of the cutting blade holder have, viewed in a cross-section transverse to the linear extension of the bearing surface, an arcuate, in particular circular arcuate, curved contour.

9. Cutting blade unit for use with a cutting blade holder of a microsurgical cutting arrangement according to one of the preceding claims, wherein the cutting blade unit comprises a cutting blade with a cutting edge formed on a first blade edge, and wherein a second blade edge of the cutting blade opposite the first blade edge forms at least two bearing points spaced apart from one another for the bearing of the cutting blade on linear bearing surface of the cutting blade holder and between each pair of adjacent bearing points is set back with respect to an imaginary straight line joining the relevant bearing points, in the direction of the first blade edge.
